(19) United States
(12) Patent Application Publication
(10) Pub. No.: US 2002/0015722 A1
(43) Pub. Date: Feb. 7, 2002

(54) Food product

(76) Inventors: Leslie John Herzog, Nyack, NY (US); Sanitha Shamanna Bhima Rao, Parsipanny, NJ (US); Matthew Richard Park, River Edge, NJ (US); John David Crump, Tenafly, NJ (US); Charles Bee Brown, Rutherford, NJ (US); Podutoori Ravinder Reddy, Columbia, MD (US); Mandy Kim Chen, Baltimore, MD (US); Matthew Patrick, Annapolis, MD (US); Michael Budd, Lincoln Park, NJ (US)

Correspondence Address: UNILEVER, PATENT DEPARTMENT, 45 RIVER ROAD, EDGEWATER, NJ, 07020, US

(21) Appl. No.: 09/564,662

(22) Filed: May 3, 2000

Related U.S. Application Data
(60) Provisional application No. 60/132,658, filed on May 5, 1999.

(51) Int. Cl. A61K 47/00; A61K 31/35; A61K 31/305

(52) U.S. Cl. 424/439; 514/456

(57) ABSTRACT

A food product containing isoflavones and flavanol is taught, where the weight ratio of isoflavones to flavanol is from 10 to 1 to 1 to 100.

### TECHNICAL FIELD OF THE INVENTION

The invention relates to food products and methods of preparation of food products. In particular to food products containing one or more isoflavone compounds.

### BACKGROUND TO THE INVENTION

Isoflavones have been proposed as ingredients for food and/or pharmaceutical products.

For example WO 98/08503 (Novogen Research) mentions the use of certain isoflavone compounds in therapeutic uses, methods, compounds, formulations, and drinks and foodstuffs.

WO 96/10341 (Schouten Industries) discloses food or health products comprising substantially pure hypocotyls of the seeds of Glycine max.

WO 93/23069 (Kelly) discloses health supplements comprising a health supplementary amount of a phyto-estrogen selected from genistein, daidzein, biochanin A, and/or formononetin.

Despite the fact that isoflavones have been suggested to have a positive effect on various health effects, the actual marketing of food products supplemented with isoflavones has up till now not taken place on a large scale.

One of the problems in formulating food products with isoflavones is that it is often difficult to find product formulations which on the one hand provide the desired health benefits and on the other hand can fairly easily be formulated at a reasonable cost. A further problem in the formulation of food products with isoflavones is that on the one hand—for cost and taste reasons—a relatively low level of supplemented ingredients is preferred while on the other hand it is desirable to have a level which leads to an appropriate bioavailability in the human body.

Furthermore consumers these days have a clear preference for food products which form part of their common food habits and which, in themselves, have a clear contribution towards a healthy diet by which no longer additives or a special diet needs to be taken.

The present invention aims to provide food products which are part of the common diet and which comprise isoflavones and flavanols in amounts sufficient to provide balanced and advantageous health benefits to consumers, especially to middle aged women aged 35 to 65. By this invention, the consumer needs not alter their daily consuming habits, while the risk towards various diseases for example osteoporosis and/or menopausal syndrome and/or hot flushes and/or cardiovascular disease and/or breast cancer and/or migraine can be reduced by the intake of the food product.

It has now been found that one or more of the above problems can be solved if a food product is prepared which contains isoflavones in combination with flavanols, whereby the ingredients are used in specific amounts.

### SUMMARY OF INVENTION

Accordingly the present invention relates to a food product comprising isoflavones and flavanol, wherein the weight ratio of isoflavones to flavanol is from 10 to 1 to 1 to 100.

The invention also relates to a method of preparing a food product whereby one or more isoflavones and one or more flavanols are included in the product.

Preferably food products of the invention are part of the normal daily diet, for example margarines or other spreads or oil based products, bakery products, dairy products e.g. yoghurt, cheese and milk-based drinks, beverages e.g. soft drinks, fruit juices and tea and coffee based drinks, sauces especially dressings and mayonnaise and confectionery products e.g. frozen confectionery products such as water-ice or ice-cream. Especially preferred are tea based products.

### DETAILED DESCRIPTION OF THE INVENTION

Isoflavones belong to the class of flavonoids. The basic formulas for isoflavones and flavanols are given in the FIG. 1. The R, R1 and R2 groups all may vary independently, but are usually hydrogen or hydroxyl. Examples of isoflavone compounds are given in WO 98/8503.

Preferred isoflavones for use in the present invention have anti-oxidant properties. In this context it is preferred that the isoflavones comprise at least one hydroxy substituent, more preferred 2 to 4 hydroxy groups. Examples of preferred isoflavones are given in formula (1) to (19) of WO 98/8503, especially preferred isoflavones are Daidzein, Genistein, dihydrodaidzein and dihydrogenistein.

Isoflavones can be produced chemically or can be isolated from natural sources e.g. plants. Especially suitable is the isolation of isoflavones from plants for example from linseed, lentils, beans, chickpeas, green peas and soya. Most preferred is to use isoflavones which have been isolated from soya.

Flavanols also belong to the class of flavonoids.

Preferred flavanols for use in the present invention have anti-oxidant properties. In this context it is preferred that the flavanols comprise at least one hydroxy substituent, more preferred 2 to 6 hydroxy groups. Examples of preferred flavanols are flava-3-ols, especially preferred are epicatechin, epicatechin gallate, epigallocatechin and epigallocatechin gallate. For the purpose of the invention the term flavanol also embraces the reaction products of naturally occurring flavanols after fermentation. For example if tea is fermented it is believed that at least part of the catechins present in the tea are oxidised, dimerised and condensed with orthoquinones leading to theaflavins and/or thearubigins. These compounds are also referred to as preferred flavanols in accordance to the invention.

Flavanols can be produced chemically or can be isolated from natural sources e.g. plants. Especially suitable is the isolation of flavanols from plants for example from grapes or tea-leaves. Most preferred is to use flavanols which have been isolated from green or black tea.

Food products according to the invention comprise isoflavones and flavanol, wherein the weight ratio of isoflavones to flavanol is from 10 to 1 to 1 to 100, more preferred 5 to 1 to 1 to 30, most preferred from 2 to 1 to 1 to 10.

Typical amounts of isoflavones in the food product can suitably be adapted to the desirable intake and the average size of a serving. Preferably the level of isoflavone is chosen such that a normal serving of the food product comprises 1 to 200 milligrams of isoflavone, more preferred 2 to 100 milligrams, especially preferred 4 to 40 milligrams, most preferred 5 to 15 milligrams. Typical sizes of a single serving are given below.

Isoflavones or flavanols may be added in their glycosylated form (which is often their natural form) or may advantageously be added as an aglycone. For the purpose of the invention the weight of the isoflavone or flavanol refers to the isoflavone or flavanol as if in aglycone form.

Typical amounts of flavanol in the food product are 0.1 to 100 times the amount of isoflavone, more preferred 0.2 to 30 times, most preferred 0.5 to 10 times. Taking into account this ratio in combination with the desirable intake of flavanol and the average size of serving the absolute level of flavanol in the food product may be determined.

Preferably the level of flavanol is chosen such that its weight ratio to the amount of isoflavone is as above and also that a normal serving of the food product comprises 1 to 1000 milligrams of flavanol, more preferred 2 to 500 milligrams, especially preferred 4 to 200 milligrams, most preferred 5 to 100 milligrams.

In this context is should be noted that some food products, e.g. tea based products already contain flavanol as one of their ingredients, these products may either already comprise the flavanol at the preferred level of to these products or flavanol will be added to increase the total level into the desired range.

The invention may usefully be applied to a variety of food products. Especially preferred is the use in food products which tend to be part of the daily diet. These products can suitably be used for repeated and regular dosage of the combination of isoflavones and flavanol over prolonged periods and therewith provide good health benefits.

Examples of preferred food products are margarines or other spreads or oil based products, bakery products, dairy products e.g. yoghurt, cheese and milk-based drinks, beverages e.g. soft drinks, fruit juices and tea and coffee based drinks, sauces especially dressings and mayonnaise and confectionery products e.g. frozen confectionery products such as water-ice or ice-cream. Especially preferred is the use in food products selected from the group of margarines and other spreads, tea based beverages, dressings and frozen confectionery products. Most preferred is the use in tea products e.g. tea based beverages or powders or leaf tea products for use in the preparation of tea based beverages.

Food products according to the invention are advantageously recommended for use by women of the age of 35 to 65. A typical recommended diet may involve the administering of 2- 100 servings of products of the invention per week, more preferred 5-50 per week. This depends on the amount of isoflavones and flavanol in the product. Especially preferred is the use of one or more products of the invention in amounts such that per week on average 20 to 1000 mg of isoflavones is consumed via products of the invention, more preferred 50 to 500 mg, most preferred 70 to 500 mg also preferable is the use of one or more products of the invention in amounts such that per week on average 20 to 1000 mg of flavanols is consumed via products of the invention, more preferred 50 to 500 mg, most preferred 70 to 500 mg. Preferable both isoflavones and flavanols are consumed at the above levels.

For margarine or other spreads the number of servings (about 14 grams) advantageously is 5- 25 per week, for example 10-20. For frozen confectionery products the number of servings (about 66 grams) advantageously is 2-14, more preferred 3-10. For tea based products (about 250 ml) the number of servings is preferably 5 to 45 per week, more preferred 10 to 30. For dressings or mayonnaise the number of servings (about 30 g)is advantageously 240 per week, more preferred 5-20 per week.

The invention will now be further illustrated by the description of tea based products in accordance to the invention. It is believed to be well within the ability of the skilled person to use the teaching provided therewith to.

### TEA BASED PRODUCTS

For the purpose of this invention the term tea based products refers to products containing tea or tea replacing herbal compositions e.g. tea-bags, leaf tea, herbal tea bags, herbal infusions, powdered tea, powdered herbal tea, ice-tea, ice herbal tea, carbonated ice tea, carbonated herbal infusions etc.

Typically some tea based products of the invention may need a preparation step shortly before consuming, e.g. the making of tea brew from tea-bags, leaf tea, herbal tea bags or herbal infusions or the solubilisation of powdered tea or powdered herbal tea. For these products it is preferred to adjust the level of isoflavones and flavanol in the product such that one serving of the final product to be consumes has the desired levels of isoflavones and flavanol as described above.

For iced tea, iced herbal tea, carbonated iced tea, carbonated herbal infusions and other ready to drink tea based products the typical size of one serving will be 250 ml or 250 grams. Preferred levels of isoflavone in these ready-to-drink products are 0.0004 to 0.1 wt %, more preferred, 0.0008 to 0.05 wt %, especially preferred 0.0016 to 0.016 wt %, most preferred, 0.002 to 0.006 wt %. Preferred levels of flavanol in these ready to drink products are 0.0004 to 0.1 wt %, more preferred, 0.0008 to 0.05 wt %, especially preferred 0.0016 to 0.016 wt %, most preferred, 0.002 to 0.006 wt %.

For products which are extracted to obtain the final product, generally the aim is to ensure that one serving of 250 ml or 250 grams comprises the desired amounts as indicated above. In this context it should be appreciated than normally only part of the isoflavones present in the tea based product to be extracted will eventually be extracted into the final tea drink. To compensate for this effect generally it is desirable to incorporate into the products to be extracted about 2 times the amount of isoflavones as is desired to have in the extract.

For leaf tea or tea-bags typically 1-5 grams of tea would be used to prepare a single serving of 250 mis. Preferred levels of isoflavone in the tea compound of such products would be 0.04 to 20 wt %, more preferred 0.08 to 10 wt %, especially preferred 0.16 to 5 wt %, most preferred 0.2 to 3 wt %.

Products would be 0.02-10 wt %, more preferred 0.04 to 5 wt %, especially preferred 0.08 to 2.5 wt %, most preferred 0.1 to 3 wt %.

If tea-bags are used the isoflavone and flavanol component may advantageously be incorporated into the tea component, however it will be appreciated that for some applications it may be advantageous to separate the isoflavone and/or the flavanol from the tea, for example by incorporating them into a separate compartment of the tea bag or applying them onto the tea-bag paper.

### EXAMPLE 1

In the following examples the source of isoflavones is SoyLife as marketed by SoyLife Nederland B.V.

The composition of SoyLife is approximately as follows:

| Ingredient | wt % |
|---|---|
| Isoflavones 1 | 3% |
| Saponins | 4% |
| Protein | 40% |
| Fat | 11% |
| Fiber | 4% |
| Ash | 5% |
| Carbohydrates | 35% |
| Cholesterol | 0% |
| Tocopherols | 0.05% |
| α-Tocopherol | 0.008% |
| Lecithin | 2% |
| Water | balance |

¹ glucosides

Novasoy obtained from Archer Daniels Midland may also be used.

The following vitamin mixes are used:

XR05837000 (ex Roche):

| Ingredient | wt % |
|---|---|
| Vitamin B6 | 2.9% |
| Vitamin B12 | 7.8% |
| Vitamin E | 72% |
| Maltodextrin | balance |

GLATT PH990097:

| Ingredient | wt % |
|---|---|
| Calciumlactate | 73.8 |
| Vitamin B6 | 0.29% |
| Vitamin B12 | 0.78% |
| Vitamin E | 7.2% |
| Maltodextrin | balance |

### EXAMPLE 2

Ice-tea mix

| Ingredient | Wt parts |
|---|---|
| MALTODEXTRIN | 37.1 |
| TEA POWDER | 8.7 |
| ASPARTAME | 2.6 |
| LEMON OIL POWDER | 0.95 |
| LEMON ESSENCE POWDER | 0.54 |
| MALIC ACID | 12.3 |
| OIL COATED MALIC ACID | 4.78 |
| MAGNESIUM OXIDE | 0.18 |
| SoyLife | 10.0 |
| VITAMIN PREMIX, = XR05837000 | 0.30 |
| CALCIUM LACTATE | 22.5 |

3.3 grams of the product can advantageously be used to prepare a serving of iced tea of 250 mis.

### EXAMPLE 3

Iced tea mix

| Ingredient | Wt parts |
|---|---|
| MALTODEXTRIN | 39.4 |
| TEA POWDER | 9.0 |
| ASPARTAME | 2.5 |
| PEACH FLAVOR | 3.6 |
| N&A APRICOT FLAVOR | 1.17 |
| CITRIC ACID | 9.05 |
| OIL COATED CITRIC ACID | 1.27 |
| MAGNESIUM OXIDE | 0.16 |
| SoyLife | 10.3 |
| VITAMIN PREMIX, = XR05837000 | 0.31 |
| CALCIUM LACTATE | 23.2 |

This mix can be used as in example 2.

### EXAMPLE 4

Flavoured tea bag

| Ingredient | parts by weight |
|---|---|
| SMOOTH BLEND TEA | 40.1 |
| HONEY FLAKES | 11.6 |
| LEMON FLAVOR | 4.0 |
| LEMON GRANULES | 3.80 |
| GLYCYRRHIZIN | 3.16 |
| HONEY GRANULE FLAVOR | 3.16 |
| SoyLife | 13.92 |
| VITAMIN/MINERAL PREMIX | 20.25 |
| PH990087 | |

4.74 grams of these are incorporated in a tea bag and used for the preparation of 250 mis of tea brew. About 50% of the soylife is extracted into the tea brew.

### EXAMPLE 5

Herbal tea bag

| Ingredient | parts by weight |
|---|---|
| HIBISCUS | 7.3 |
| CHAMOMILE | 31.7 |
| CHICORY | 5.7 |
| PEACH FLAVOR GRANULES | 3.17 |
| APRICOT FLAVOR GRANULES | 0.63 |
| SoyLife | 20.95 |
| VITAMIN/MINERAL PREMIX | 30.48 |
| PH990087 | |

3.15 grams of these are incorporated in a tea bag and used for the preparation of 250 mls of tea brew. About 50% of the soylife is extracted into the tea brew. An appropriate amount of flavanol is also added.

### EXAMPLE 6

Herbal tea bags

| Ingredient | parts by weight |
|---|---|
| ROSEHIPS | 9.6 |
| HIBISCUS | 26.4 |
| CHICORY ROOT | 7.21 |
| LICORICE ROOT | 0.72 |
| CITRIC ACID | 1.68 |
| ORANGE/PINEAPPLE/MANGO | 3.85 |
| ORANGE | 4.81 |
| ALMOND GRANULES | 0.96 |
| CINNAMON | 2.88 |
| ALLSPICE | 2.88 |
| SoyLife GRANULAR | 15.87 |
| VITAMIN/MINERAL PREMIX | 23.08 |
| PH990087 | |

4.16 grams of the tea is incorporated in a tea bag and used to prepare 250 mls of tea brew. An appropriate amount of flavanol is added.

### EXAMPLE 10

A tea formula similar to that of Example 4 is compared to a standard.

| | Control | VI A. |
|---|---|---|
| Smooth blend tea | 1.9 | 1.9 |
| Honey flakes | .55 | .55 |
| Lemon flavor | — | .19 |
| Lemon granules | .18 | .18 |
| Glycyrrhizin | .15 | .15 |
| Honey granules flavor | — | .16 |
| SoyLife | .66 | .66 |
| Vitamin pre mix* | .96 | .96 |
| | 4.40 g | 4.75 g |

*Glatt PH990097
Calcium lactate 0.74
Vitamin mix 0.1

Testing is conducted in-house with 50 employee users of flavored tea. Samples are evaluated hot. Panelists rate each sample on a 9 point hedonic scale for overall acceptability and select a preferred sample. No significant acceptability differences are detected. Both samples are rated slightly above “Like Slightly”. However, the isoflavone containing prototype, which also contains additional flavor, was significantly preferred over current Honey & Lemon (p=0.05)

### Sensory Methods:

Sensory Approach

An in-house acceptability and preference test is conducted with a panel of 50 employee users of flavored tea who are acceptors of honey and lemon flavors. Panelists are requested to rate each sample on a 9 point hedonic scale and select a preferred sample.

Sample Presentation

Samples are brewed for 3 minutes, and served at approximately 150° F., in labeled white Corelle cups under white lights in the Sensory booths, using balanced orders of presentation.

### Results

No significant acceptability differences are found between samples, although the isoflavone sample contained more flavoring. The isoflavone prototype was significantly preferred over current production Honey & Lemon Flavored Tea.

TABLE I
Current Product vs. Isoflavone Containing Prototype
Acceptability/Preference
(N = 50)

| | Mean Rating | Number | |
|---|---|---|---|
| Sample | Overall Liking | Preferring | % |
| Project Eve Prototype | 6.3 | 32a* | 64 |
| Honey & Lemon | | | |
| Benchmark sample | 6.3 | 18b | 36 |
| Current Honey & Lemon | | | |

*Values followed by a different letter are significantly different at p = 0.05.

Acceptability Rating Scale

| | | | | | | | | |
|---|---|---|---|---|---|---|---|---|
| 1 | 2 | 3 | 4 | 5 | 6 | 7 | 8 | 9 |
| Dislike | Dislike | Dislike | Dislike | Neither Like | Like | Like | Like | Like |
| Extremely | Very Much | Moderately | Slightly | nor Dislike | Slightly | Moderately | Very Much | Extremely |

It is understood that the examples and embodiments described herein are for illustrative purposes only and that various modifications or changes in the light thereof will be suggested to persons skilled in the art and are to be included within the spirit and purview of this application and the scope of the appended claims.

1. A food product comprising isoflavones and flavanol, wherein the weight ratio of isoflavones to flavanol is from 10 to 1 to 1 to 100.

2. Food product according to claim 1, being part of the normal daily diet.

3. Food products according to claim 1 wherein the food product is selected from the group consisting of margarines or other spreads, tea based products, dressings, mayonnaise and frozen confectionery products.

4. Food product according to claim 1, wherein the food product is a tea based product.

5. Food product according to claim 1, wherein the weight ratio of isoflavone to flavanol is from 2 to 1 to 1 to 10.

6. Food product according to claim 1, wherein the level of isoflavone is chosen such that a normal serving of the food product comprises 1 to 200 milligrams of isoflavone.

7. Food product according to claim 1, wherein the level of flavanol is chosen such a normal serving of the food product comprises 1 to 200 milligram of flavanol.

8. Food product according to claim 1, which is a tea based beverage wherein the isoflavone level is 0.0004 to 0.1 wt % and the flavanol level is 0.0004 to 0.1 wt %.

9. Food product according to claim 1 which is a tea-leaf or tea-bag product comprising leaf tea wherein the isoflavone level is 0.04 to 20 wt % and the flavanol level is 0.04 to 20 wt % based on the weight of the tea present in the tea-leaf or tea-bag.

10. A method for the prevention or reduction of symptoms of osteoporosis and/or menopausal syndrome and/or hot flushes and/or cardiovascular disease and/or breast cancer and/or migraine which comprises administering to a subject an effective amount of a food product according to claim 1.

11. A method according to claim 13, wherein the subject is a female aged 35 to 65 years.

12. A method according to claim 13, wherein the amounts and types of products are chosen that per week on average 20 to 1000 mg of isoflavones is consumed via products according to claim 1.

13. Use of isoflavones and flavanol in a weight ratio of isoflavones to flavanol is from 10 to 1 to 1 to 100 in the preparation of a food product for the prevention or reduction of symptoms of osteoporosis and/or menopausal syndrome and/or hot flushes and/or cardiovascular disease and/or breast cancer and/or migraine.

14. Use according to claim 13, wherein food product is intended to be administered to females of 35 to 65 years.

15. Use according to claim 13, wherein the food product is a tea based product.
